(19) United States
(12) Patent Application Publication
(10) Pub. No.: US 2012/0264948 A1
(43) Pub. Date: Oct. 18, 2012

(54) MEMBRANE SEPARATION AND PURIFICATION OF MONATIN

(75) Inventors: Brent Howard Hilbert, Annandale, MN (US); Trent H. Pemble, St. Bonifacius, MN (US); Michael A. Porter, Maple Gove, MN (US); Christopher Solheid, Minneapolis, MN (US)

(73) Assignee: Cargill, Incorporated, Wayzata, MN (US)

(21) Appl. No.: 13/518,965

(22) PCT Filed: Dec. 30, 2010
(86) PCT No.: PCT/US10/62600

§ 371 Date: Jun. 25, 2012

Related U.S. Application Data
(60) Provisional application No. 61/335,022, filed on Dec. 30, 2009.

(51) Int. Cl. C07D 209/20

(52) U.S. Cl. 548/495

(57) ABSTRACT

Methods and systems for separating and purifying monatin are described. In the production of monatin, a mixture is formed which includes monatin, starting materials and intermediates. The methods and systems include a membrane having a zeta potential of from about −19 to −6 such that the membrane rejects greater than about 90% of the monatin. In some embodiments, the membrane is a nanofiltration membrane.

### CROSS-REFERENCE TO RELATED APPLICATIONS

This application claims priority to U.S. provisional application Ser. No. 61/335,022, filed 30 Dec. 2009, entitled MEMBRANE SEPARATION AND PURIFICATION OF MONATIN, which is incorporated herein by reference in its entirety.

### FIELD

The present disclosure relates generally to a method and system for producing monatin. In particular, the present disclosure relates to a method and system for separating and purifying monatin from a mixture comprising monatin, starting materials and intermediates.

### BACKGROUND

Monatin (2-hydroxy-2-(indol-3-ylmethyl)-4-aminoglutaric acid) is a naturally occurring, high intensity or high potency sweetener that was originally isolated from the plant Sclerochiton ilicifolius, found in the Transvaal Region of South Africa. Monatin has the chemical structure:

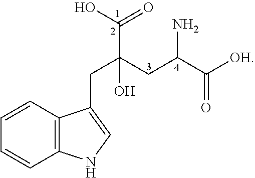

Because of various naming conventions, monatin is also known by a number of alternative chemical names, including: 2-hydroxy-2-(indol-3-ylmethyl)-4-aminoglutaric acid; 4-amino-2-hydroxy-2-(1H-indol-3-ylmethyl)-pentanedioic acid; 4-hydroxy-4-(3-indolylmethyl)glutamic acid; and 3-(1-amino-1,3-dicarboxy-3-hydroxy-but-4-yl)indole.

Monatin has two chiral centers thus leading to four potential stereoisomeric configurations: the R,R configuration (the “R,R stereoisomer” or “R,R monatin”); the S,S configuration (the “S,S stereoisomer” or “S,S monatin”); the R,S configuration (the “R,S stereoisomer” or “R,S monatin”); and the S,R configuration (the “S,R stereoisomer” or “S,R monatin”).

Reference is made to WO 2003/091396 A2, which discloses, inter alia, polypeptides, pathways, and microorganisms for in vivo and in vitro production of monatin. WO 2003/091396 A2 (see, e.g., FIGS. 1-3 and 11-13) and U.S. Patent Publication No. 2005/282260 describe the production of monatin from tryptophan through multi-step pathways involving biological conversions with polypeptides (proteins) or enzymes. One pathway described involves converting tryptophan to indole-3-pyruvate (“I3P”) (reaction (1)), converting indole-3-pyruvate to 2-hydroxy2-(indol-3-ylmethyl)-4-keto glutaric acid (monatin precursor, “MP”) (reaction (2)), and converting MP to monatin (reaction (3)). The three reactions can be performed biologically, for example, with enzymes.

### SUMMARY

One embodiment is directed toward a method of recovering monatin from a mixture including monatin and at least one of I3P, tryptophan, pyruvate and monatin precursor, comprising pumping the mixture across a membrane having a zeta potential of from about −19.0 to −6.0 and wherein greater than about 95% of the monatin is rejected by the membrane. In one aspect, the mixture includes monatin and I3P and the membrane produces an α value greater than 15 or greater than 30 for I3P at operating conditions of 15° C. and 30° C. respectively. In a further aspect, the a value for I3P is from about 15.5 to 18.4 at 15° C. In another aspect, the a value is from about 33.2 to 42.7 at 30° C.

The details of one or more non-limiting embodiments of the invention are set forth in the description below. Other embodiments of the invention should be apparent to those of ordinary skill in the art after consideration of the present disclosure.

### BRIEF DESCRIPTION OF THE DRAWINGS

FIG. 1 is a block diagram of an exemplary system for the separation and purification of monatin from a mixture including monatin, starting materials and intermediates.

### DETAILED DESCRIPTION

The present disclosure is directed to a method and system for separating and purifying monatin from a mixture including monatin, starting materials used in the production of monatin, and intermediates formed during the production of monatin. The method and system includes a chromatography unit that is packed with a reverse phase resin that is effective at separately eluting monatin from the other components in the mixture. The eluted monatin has a purity of at least 90%. In some embodiments, the eluted monatin is a stereoisomerically-enriched R,R monatin. In some embodiments, the chromatography unit is a dynamic axial compression (DAC) column. In some embodiments, the reverse phase resin is formed from polystyrene-divenylbenzene.

Monatin has an excellent sweetness quality, and depending on a particular composition, monatin may be several hundred times sweeter than sucrose, and in some cases thousands of times sweeter than sucrose. As stated above, monatin has four stereoisomeric configurations. The S,S stereoisomer of monatin is about 50-200 times sweeter than sucrose by weight. The R,R stereoisomer of monatin is about 2000-2400 times sweeter than sucrose by weight. As used herein, unless otherwise indicated, the term “monatin” is used to refer to compositions including any combination of the four stereoisomers of monatin (or any of the salts thereof), including a single isomeric form.

Monatin may be synthesized in whole or in part by one or more of a biosynthetic pathway, chemically synthesized, or isolated from a natural source. If a biosynthetic pathway is used, it may be carried out in vitro or in vivo and may include one or more reactions such as the equilibrium reactions provided below as reactions (1)-(3). In one embodiment, is a biosynthetic production of monatin via enzymatic conversions starting from tryptophan and pyruvate and following the three equilibrium reactions below:

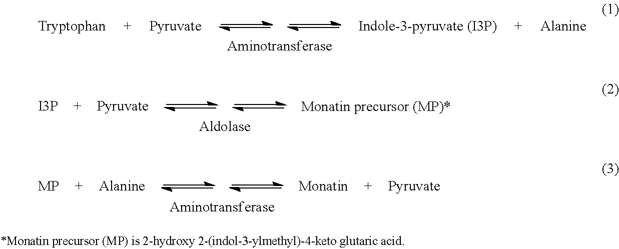

The following side-reactions may also occur, resulting in production of hydroxymethyl-oxo-glutarate (HMO), hydroxymethylglutamate (HMG) or a combination thereof:

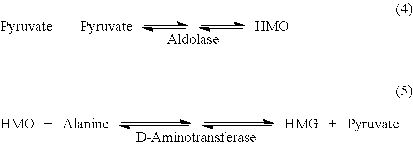

In the pathway shown above, in reaction (1), tryptophan and pyruvate are enzymatically converted to indole-3-pyruvate (I3P) and alanine in a reversible reaction. As exemplified above, an enzyme, here an aminotransferase, is used to facilitate (catalyze) this reaction. In reaction (1), tryptophan donates its amino group to pyruvate and becomes I3P. In reaction (1), the amino group acceptor is pyruvate, which then becomes alanine as a result of the action of the aminotransferase. The amino group acceptor for reaction (1) is pyruvate; the amino group donor for reaction (3) is alanine. The formation of indole-3-pyruvate in reaction (1) can also be performed by an enzyme that utilizes other α-keto acids as amino group acceptors, such as oxaloacetic acid and α-keto-glutaric acid. Similarly, the formation of monatin from MP (reaction (3)) can be performed by an enzyme that utilizes amino acids other than alanine as the amino group donor. These include, but are not limited to, aspartic acid, glutamic acid, and tryptophan.

Some of the enzymes useful in connection with reaction (1) may also be useful in connection with reaction (3). For example, aminotransferase may be useful for both reactions (1) and (3). The equilibrium for reaction (2), the aldolase-mediated reaction of indole-3-pyruvate to form MP (i.e. the aldolase reaction), favors the cleavage reaction generating indole-3-pyruvate and pyruvate rather than the addition reaction that produces the alpha-keto acid precursor to monatin (i.e. MP). The equilibrium constants of the aminotransferase-mediated reactions of tryptophan to form indole-3-pyruvate (reaction (1)) and of MP to form monatin (reaction (3)) are each thought to be approximately one. Methods may be used to drive reaction (3) from left to right and prevent or minimize the reverse reaction. For example, an increased concentration of alanine in the reaction mixture may help drive forward reaction (3). Reference is made to US Publication No. 2009/0198072 (application Ser. No. 12/315,685), which is also assigned to Cargill, the assignee of this application.

The overall production of monatin from tryptophan and pyruvate is referred to herein as a multi-step pathway or a multi-step equilibrium pathway. A multi-step pathway refers to a series of reactions that are linked to each other such that subsequent reactions utilize at least one product of an earlier reaction. In such a pathway, the substrate (for example, tryptophan) of the first reaction is converted into one or more products, and at least one of those products (for example, indole-3-pyruvate) can be utilized as a substrate for the second reaction. The three reactions above are equilibrium reactions such that the reactions are reversible. As used herein, a multi-step equilibrium pathway is a multi-step pathway in which at least one of the reactions in the pathway is an equilibrium or reversible reaction.

Because the R,R stereoisomer of monatin is the sweetest of the four stereoisomers, it may be preferable to selectively produce R,R monatin. For purposes of this disclosure, the focus is on the production of R,R monatin. However, it is recognized that the present disclosure is applicable to the production of any of the stereoisomeric forms of monatin (R,R; S,S; S,R; and R,S), alone or in combination.

In some embodiments, the monatin consists essentially of one stereoisomer—for example, consists essentially of S,S monatin or consists essentially of R,R monatin. In other embodiments, the monatin is predominately one stereoisomer—for example, predominately S,S monatin or predominately R,R monatin. “Predominantly” means that of the monatin stereoisomers present in the monatin, the monatin contains greater than 90% of a particular stereoisomer. In some embodiments, the monatin is substantially free of one stereoisomer—for example, substantially free of S,S monatin. “Substantially free” means that of the monatin stereoisomers present in the monatin, the monatin contains less than 2% of a particular stereoisomer. In some embodiments, the monatin is a stereoisomerically-enriched monatin mixture. “Stereoisomerically-enriched monatin mixture” means that the monatin contains more than one stereoisomer and at least 60% of the monatin stereoisomers in the mixture is a particular stereoisomer. In other embodiments, the monatin contains greater than 60%, 65%, 70%, 75%, 80%, 85%, 90%, 95%, 98% or 99% of a particular monatin stereoisomer. In another embodiment, a monatin composition comprises a stereoisomerically-enriched R,R-monatin, which means that the monatin comprises at least 60% R,R monatin. In other embodiments, stereoisomerically-enriched R,R-monatin comprises greater than 60%, 65%, 70%, 75%, 80%, 85%, 90%, 95%, 98% or 99% of R,R monatin.

For example, to produce R,R monatin using the three-step pathway shown above (reactions (1)-(3)), the starting material may be D-tryptophan, and the enzymes may be a D-aminotranferase and an R-specific aldolase. The three reactions, which are shown below, may be carried out in a single reactor or a multiple-reactor system.

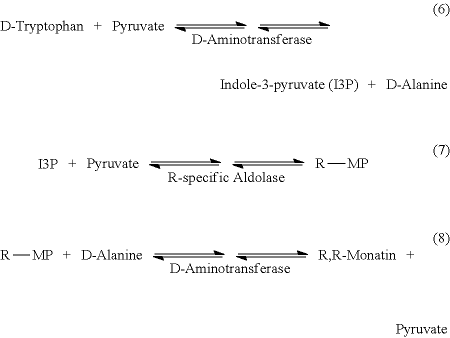

In an embodiment in which a single reactor is used, the two enzymes (i.e. the D-aminotransferase and the R-specific aldolase) may be added at the same time and the three reactions may run simultaneously. The same enzyme may be used to catalyze reactions (6) and (8). A D-aminotransferase is an enzyme with aminotransferase activity that selectively produces, in the reactions shown above, D-alanine and R,R-monatin. An R-specific aldolase is an enzyme with aldolase activity that selectively produces R-MP, as shown in reaction (7) above. Although a focus in the present disclosure is on R,R monatin, it is recognized that the method and system of separating and purifying monatin is applicable to any of the stereoisomeric forms of monatin.

There are multiple alternatives to the above pathway (i.e. reactions (6)-(8)) for producing R,R-monatin. For example, L-tryptophan may be used as a starting material instead of D-tryptophan. In that case, an L-aminotransferase may be used to produce indole-3-pyruvate and L-alanine from L-tryptophan. Because L-alanine is produced, this pathway may require the use of an alanine racemase to convert the L-alanine to D-alanine, thus adding a fourth reaction to the monatin production pathway. (D-alanine is required to produce R,R monatin from the R-stereoisomer of monatin precursor (R-MP). In addition to requiring another enzyme (alanine racemase), undesired side reactions may also occur in this pathway. For example, L-alanine may react with the L-aminotransferase to produce R,S-monatin, or D-alanine may react with I3P to form D-tryptophan, resulting in a racemate of L-tryptophan and D-tryptophan, which has poor solubility. Some disadvantages of this pathway may be avoided by using a two reactor system as opposed to a single reactor system. It is recognized that there are additional alternatives not specifically disclosed herein for performing the three-step equilibrium pathway to produce monatin. The method and system described herein for separating and purifying monatin is applicable to monatin produced using alternative pathways to what is disclosed herein.

As described above, in some pathways, it may be preferable to perform the monatin producing reactions in two or more separate reactors, while in other pathways it may be preferable to use a single reactor system. The decision to use a one reactor or a multiple reactor system may depend, in part, on whether D-tryptophan or L-tryptophan is used as a starting material. A single reactor system is obviously simpler in design, eliminating the need for a second reactor, as well as eliminating, in some cases, a need for a separation step between the first and second reactors. It is recognized that the method and system described herein for separating and purifying monatin may be used in combination with both a single reactor system and a multiple reactor system for the production of monatin.

Although the present disclosure focuses on the production of monatin using the biosynthetic multi-step equilibrium pathway described above, monatin may also be produced chemically or using a combination of both chemical synthesis and an enzymatic pathway. Regardless of the method used to produce monatin, the resulting monatin may be present in a mixture that contains other components, including starting materials, intermediates, side products of the monatin-producing reactions or combinations thereof. It is preferable to separate the monatin from these other components, which may include, for example, tryptophan, pyruvate, alanine, I3P, MP, HMG and HMO. Thus, one aspect of the present disclosure is a method and system for separating and purifying monatin from this mixture using nanofiltration, chromatography or a combination thereof, such that the monatin has at least 90% purity.

After producing monatin, using either a single reactor or multiple reactor system, the monatin-containing mixture may undergo one or more intermediate processing steps before the chromatography process described herein. For example, the monatin-containing mixture may undergo a filtration process (also referred to herein as ultrafiltration or ultrafiltration process), upstream of the chromatography unit, which is designed to remove enzymes in the monatin-containing mixture. Other means of removing the enzymes would also be acceptable. Another membrane filtration process (referred to hereinafter as the nanofiltration process, nanofiltration, membrane filtration process or membrane process), also located upstream of the chromatography unit, may be used to remove some of the intermediates, such as I3P and tryptophan, from the monatin-containing mixture, thus making the chromatography process more efficient. The nanofiltration process may also be used to concentrate permeate collected from the ultrafiltration process. In one aspect, enough water is removed to create a solution that is six times more concentrated than the original permeate from the ultrafiltration process. A further benefit of the nanofiltration process is that some of the other reaction components and intermediates are removed, allowing for stronger monatin peak resolution during operation of the chromatography unit. In one aspect, about 80% of pyruvate, about 30% tryptophan and almost about 50% of the I3P and alanine are removed from the concentrate.

A purpose of the method and system described herein is to recover as close to 100% as possible of the monatin produced, at a high purity level. It is recognized that although it may be possible to elute essentially all of the monatin from the chromatography column, if the monatin is not “pure” monatin, it is not defined herein as “recovered” monatin. As used herein, “pure” monatin is defined as a composition containing at least 90% by weight monatin, which is defined on a dry weight basis and corrected for inorganic counter ions and which is may also be referred to as having 90% purity or a purity of 90%. In some embodiments, the purity may be at least 95%; in other embodiments, at least 96%, at least 97%, at least 98%, and at least 99%.

Recovery is defined herein as the amount of pure monatin that is recovered from the mixture based on the starting mass of monatin. In some embodiments, about 80% by weight of the monatin, also on a dry weight basis, is recovered from the monatin-containing mixture. It is recognized that, in other embodiments, the system may be designed to recover less than 80% by weight of the monatin and/or recover monatin having a purity of less than 90%. It may be more efficient to recover less than 80%, depending, for example, on an overall system design for monatin production which includes recycle streams.

The separation and purification process described herein is designed, in large part, to maximize an overall carbon conversion efficiency within the monatin production system. Accordingly, in one embodiment the reported separation and purification process is designed to maximize recovery of monatin relative to the mass of monatin injected into the system. As used herein “carbon” may refer to any of the starting materials, intermediates, or products in the monatin-producing pathway.

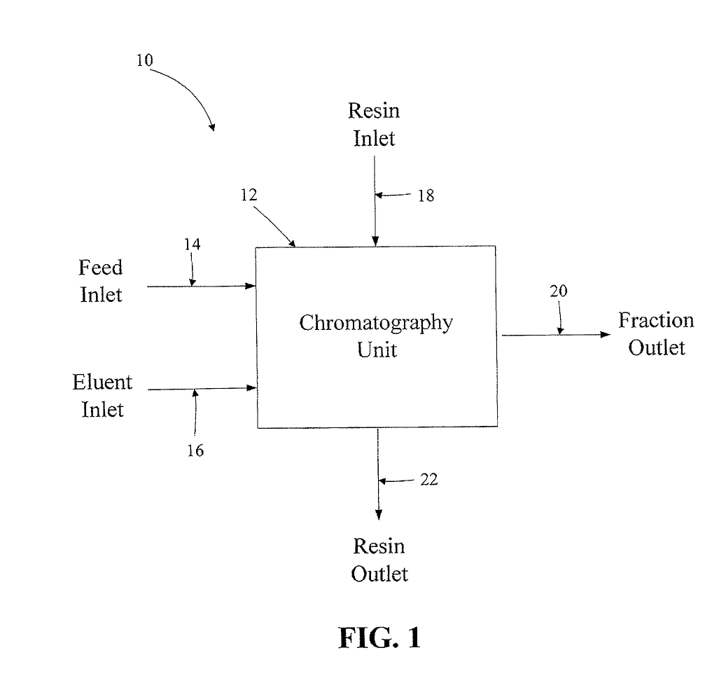

FIG. 1 is a block diagram of an exemplary system for the separation and purification of monatin. System 10 includes chromatography unit 12, feed inlet 14, eluent inlet 16, resin inlet 18, fraction outlet 20 and resin outlet 22. In some embodiments, chromatography unit 12 is located downstream of an enzyme removal unit. Chromatography unit 12 is a column packed with resin that forms a stationary phase in the chromatography separation process. The column is packed by loading resin into the column through resin inlet 18. At the end of operation, the resin may be removed from the column through resin outlet 22. The feed material (i.e. the monatin-containing mixture) is injected into the column through feed inlet 14. The monatin-containing mixture includes tryptophan, pyruvate, monatin, MP, I3P, alanine, HMO and HMG. The components in the monatin-containing mixture are adsorbed by the packed resin in the column. A mobile phase (an eluent) passes through the column through eluent inlet 16 and is designed to elute the components from the column through fraction outlet 20.

In some embodiments, the eluent is a mixture of water and an organic solvent that is miscible with water. These organic solvents may include but are not limited to acetonitrile, methanol or tetrahydrofuran, ethanol and 2-propanol (iso-propyl alcohol). The organic solvents may also include ethanol, isopropranol, methanol and mixtures thereof. In some embodiments, the eluent in system 10 is essentially all water, with no organic solvent.

A pump is used to inject the monatin-containing mixture into chromatography unit 12. The resin inside chromatography unit 12 causes the various components in the mixture to adsorb to the resin particles based on each component's affinity for the resin. The eluent is then pumped into chromatography unit 12 through eluent inlet 16. As the eluent passes through the column the components adsorbed by the resin in the column are eluted and flow out through the column with the eluent via fraction outlet 20. The most weakly adsorbed components (those with the lowest affinity for the resin) elute first. The most strongly adsorbed (i.e. highest affinity) elute last. The components may thus be separated by taking different fractions from the column. This may be done, for example, by transferring the outlet stream from fraction outlet 20 into a different container for each fraction. The fractionations from the column are described in further detail below in reference to FIG. 3. Depending on the volume of the monatin-containing mixture, multiple injections of the monatin-containing mixture through feed inlet 14 may be necessary.

In some embodiments, chromatography unit 12 uses reversed phase chromatography, meaning that the stationary phase or resin is non-polar. As compared to “normal” chromatography which uses a hydrophilic surface chemistry having a stronger affinity for polar compounds, in reversed phase chromatography the elution order of the components is reversed. The polar compounds are eluted first while non-polar compounds are retained. Thus the resin in reversed phase chromatography may be any inert non-polar substance; however, the particular composition of the resin directly impacts the separation behavior of the monatin-containing mixture, and small changes in the surface chemistries of the resin may lead to important changes in selectivity. When reversed phase chromatography is used to separate monatin from the other components in the monatin-containing mixture, the more polar compounds (e.g. pyruvate, alanine, HMO and HMG) are eluted first and are usually eluted closely together. The remaining four components elute more separately and in the following sequence—MP, monatin, I3P, and tryptophan.

The number of fractions taken from the column may depend in part on the selectivity of the resin to separate MP and monatin and to separate I3P and monatin. The number of fractions may also depend on whether any of the fractions are intended to be recycled back to other processing units with the monatin production system. In some embodiments, the first fraction from the chromatography column may include HMO, HMG, alanine and pyruvate. As stated above, these polar compounds usually elute closely together if a reversed phase resin is used in chromatography unit 12. In some embodiments, at least one fraction from the column contains monatin having a purity of at least 90%. In other embodiments, at least one fraction from the column contains monatin having a purity of at least 95%.

As mentioned above, the eluent in system 10 of FIG. 1 may be a mixture of water and an organic solvent (for example, ethanol), or the eluent may be essentially all water. A major advantage of using essentially all water as the eluent is simplification of operating system 10. When the eluent includes ethanol, an evaporation step is required downstream of chromatography unit 12 to remove the ethanol. This extra step may increase production costs and require additional environmental, health and safety precautions.

In some embodiments, chromatography unit 12 utilizes dynamic axial compression (DAC) technology in combination with the resin described above. An example of a DAC column that may be used is a ProChrom column from Novasep (Pompey, France).

As described above, in reversed phase chromatography, the less polar components (MP, monatin, I3P and tryptophan) are eluted later. More specifically, tryptophan is the last component to be eluted due to its high affinity for the resin, and often tryptophan is eluted much later than its nearest component, I3P. As stated above, in some embodiments, the eluent is pumped through the column in two stages—with the first stage being a down flow and the second stage pumping the eluent in the opposite direction (up flow). This may be done, in part, to better elute the tryptophan. In other embodiments, instead of switching the direction of flow of the eluent, an all water eluent may be used in a first elution stage and a water/ethanol eluent may be used in the second stage in order to elute tryptophan.

In some embodiments using a DAC column in combination with a reverse phase resin, the following operating conditions may be used: An oxygen free environment is maintained due to instability of one or more intermediates (for example, I3P) in the presence of oxygen. As such, before starting the DAC column, the feed and elution tanks may be sparged with nitrogen and then during operation, it may be kept under a nitrogen overlay. The temperature inside the DAC column may be maintained at an operating temperature between about 10 and about 30 degrees Celsius. In some embodiments, the temperature may be maintained at less than about 25 degrees Celsius. In other embodiments, the temperature is maintained between about 10 and about 18 degrees Celsius; in yet other embodiments, the temperature is maintained at about 15 degrees Celsius. The pH inside the DAC column prior to injection may be maintained between about 6.0 and about 9.0 depending, in part, on the pH and ionic strength of the eluent chosen.

As described above, intermediate processing units may be included in the monatin-producing system and located between the bioreactor and the chromatography unit. Depending on their existence and design, these other processing units may likely effect a composition of the input stream or feed material to the chromatography column. Without an intermediate processing step, there may be a high level of intermediates, impurities or both (for example, I3P) in the feed material injected into the chromatography column. In some embodiments, a ratio of monatin to I3P entering the column is less than about 1; in other embodiments, the ratio is less than about 0.5. Even at ratios of less than 0.5 (where there is a significant amount of I3P relative to monatin), the DAC column and the reverse phase resin disclosed herein make it feasible to recover monatin having at least 90% purity.

On the other hand, it is recognized that using another processing unit between the bioreactor and the chromatography column, to reduce the amount of one or more components of the monatin-containing mixture, including water, may increase the efficiency of the chromatography column. The increased efficiency may result in part from the fact that while the same amount of a particular component is present in a mixture, the overall volume of the mixture is reduced. In an embodiment, a DAC chromatography column is used in combination with a membrane located upstream of the chromatography column and configured to reduce the relative proportion of at least one or more of the intermediates, impurities or both in the mixture prior to adding the mixture to the chromatography column. Thus, in one aspect, the monatin purity of the column feed is increased prior to separation thereby increasing the efficiency of the chromatographic separation. In some embodiments, and as is more fully described below, the membrane is a nanofiltration membrane having a zeta potential of from about −19.0 to −6.0 and monatin rejection values greater than 95%, greater than 96%, greater than 97% and in some instances greater than 98%. In an alternative embodiment, a reverse osmosis membrane may be used. A benefit of using a reverse osmosis membrane is that it is adapted to remove water and as such increase the concentration of the feed material. In one aspect, the reverse osmosis membrane will not greatly affect the monatin purity of the solution unlike the nanofiltration membrane which produces a solution having increased monatin purity at a higher than original concentration.

Nanofiltration is a pressure driven membrane separation process. The filtration generally takes place on a selective separation layer on an organic semi-permeable membrane. The driving force of the nanofiltration is the pressure difference between the feed (retentate) and the filtrate (permeate) side at the separation layer of the membrane or more precisely the Trans Membrane Pressure (TMP). Due to the selectivity of the membrane, however, one or several components of a dissolved mixture may be retained by the membrane despite the driving force, while water and substances with low molecular weight (e.g. <about 200 Daltons) may be able to permeate the semi-permeable separation layer. In part because nanofiltration membranes may also have a selectivity for the polarity or charge of the dissolved components, it is generally possible for larger molecules with the appropriate polarity or charge to pass through the membrane while smaller molecules with the appropriate polarity or charge are retained. As such, these membranes typically operate selectively in separating materials and selection may be due to more than the physical size (as measured for instance by molecular weight) of the components to be separated.

In one aspect, a nanofiltration membrane is provided in order to remove water and retain monatin. A target is to remove enough water to reach a concentration as high as possible without creating insolubles (e.g. tryptophan) to feed the subsequent process step. One option, when operating at 25° C., is to create a solution that is about six times more concentrated than the original permeate from the ultrafiltration process. At 25° C., a concentration factor of six may be selected in part because at higher concentration factors tryptophan could precipitate, which may consequently and negatively influence operation of the membrane through blockage or destruction for example. In another aspect, a non-soluble solution may have negative influences on other processing steps as well such as reverse phase separation. Furthermore, at higher concentration factors there may be loss of permeate flux and/or loss of carbon. The concentration factor may be dependent on the size of the membrane as well as the operating temperature. Thus, the concentration factor may require modification if operated in different temperature ranges. The concentration factor may also vary depending on the size of the membrane and pressure of operation.

Due in part to the fact that the components in the solution have different molecular weights, an additional benefit of using a membrane process is that some of the non-monatin components are at least partially removed. In one aspect, this may allow for better subsequent processing downstream. For instance, by using a membrane upstream of the chromatography column, one or more of the components in the monatin-containing mixture may be reduced. For example, the membrane may be used to reduce concentration of I3P and/or tryptophan in the mixture added to the chromatography column. In some embodiments, a ratio of monatin to I3P in the mixture entering the chromatography column is at least about 2; in other embodiments, at least about 3; and in yet other embodiments, at least about 5. Because the mixture entering the chromatography column has a reduced concentration of non-monatin components, in some embodiments, tryptophan is eluted from the mixture in fewer bed volumes, thus requiring less eluent and resulting in a faster elution time. In addition, there may be a particular benefit of reducing the concentration of I3P in the mixture entering the chromatography column because I3P elutes close to monatin during chromatography, making it difficult to obtain a higher purity monatin. Further, I3P is a less preferred component to be present in the final product because it is susceptible to oxidation and decomposition into a pinkish byproduct either in the final monatin or in subsequent applications of the monatin. It should be noted and as one of skill in the art would recognize that although the concentration of a compound may actually rise during treatment, the percentage of the compound in the whole will actually drop. There are several factors to consider when selecting a membrane. Common factors include membrane composition, molecular weight cutoff, wetability, salt rejection and baseline flux. Some additional factors factors include selectivity of the membrane, membrane life, the ability to obtain high concentration factors, and the ability to clean the membrane and recover the flux and or permeability of the membrane, as well as selecting a membrane that does not chemically impact the product to name a few. It was discovered, however, that the membrane specifications alone were not ultimately determinative of the ability to remove components such as water, I3P, and potentially tryptophan, from a mixture containing monatin, water, I3P and tryptophan. A variety of different membranes were tested with the main target of removing water in the permeate and retaining monatin in the retentate. Surprisingly, and as is demonstrated by the values listed in Tables 1a-b, some of the membranes produced alpha (α) values greater than 30 for I3P, where a high alpha value is generally desired and calculated as:

α=(CR₀/CP₀)/(CRᵢ/CPᵢ)

where CR₀ is Concentration of monatin in retentate; CP₀ is Concentration of monatin in the permeate; CRᵢ is Concentration of a certain non-monatin in retentate; and CPᵢ is Concentration of a certain non-monatin in the permeate.

This was particularly observed for Test ID B11 and Test ID B19. Transmembrane pressure (TMP), which is calculated as:

TMP=(Feed Pressure+Discharge Pressure)/2−Permeate Pressure

is one determinate of alpha value. For each of the membranes having a test ID starting with the letter “A” in Table 1a, the feed pressure was about 140 psi and the TMP was 100 at temperature of 15° C. For each of the membranes having a test ID starting with the letter “B” in Table 1a, the feed pressure was about 140 psi and the TMP was 100 at temperature of 15° C. The data reported in table 1b was conducted at a feed pressure of about 220 psi and a TMP of 170 at temperature of 30° C. Although the process was conducted at a TMP of 100, one of ordinary skill in the art would recognize the benefits of operating at higher pressures to optimize the process for more concentrated mixtures being passed through the membrane; for example to maintain a desired flux rate across the membrane and/or to maintain retention of components within the mixture.

TABLE 1a
Membrane Performance at 15° C.

| | | | | | Water | alpha (15C) | | | | | | | |
|---|---|---|---|---|---|---|---|---|---|---|---|---|---|
| | | | | | Flux | | | | | | | | Monatin |
| Test ID | Manufacturer | Membrane | MWCO | Type/Material | (LMH/psi) | I3P | MP | Trp | Ala | Pyr | HMO | HMG | Rej(%) |
| A3 | Sepro | NF2 | | TFC | 1.395 | 2.14 | 0.71 | 2.17 | 2.71 | 3.12 | 0.76 | 1.09 | 72.66 |
| B11 | Sepro | NF5 | | TFC | 0.976 | 18.32 | 0.87 | 5.78 | 10.53 | 34.29 | 0.94 | 1.98 | 96.99 |
| B18 | GE | HL | 150~300 | TF | 0.880 | 4.08 | 0.75 | 2.34 | 2.97 | 6.51 | 0.82 | 1.70 | 85.83 |
| B19 | GE | DL | 150~300 | TF | 0.459 | 15.59 | 0.84 | 5.65 | 10.17 | 35.22 | 0.96 | 1.49 | 96.51 |
| A1 | Koch | SR3 | 200 | TFC | 0.766 | 1.22 | 0.79 | 1.24 | 1.30 | 1.39 | 0.88 | 1.03 | 39.83 |
| A2 | DOW | NF | | TFC/ | 0.445 | 1.84 | 0.81 | 1.98 | 2.35 | 2.67 | 0.87 | 1.05 | 65.14 |
| | | | | Polypiperazine | | | | | | | | | |
| | | | | amide | | | | | | | | | |
| A5 | Hydranautics | ESNA1- | 270 | Composite | 5.142 | 1.09 | 0.84 | 1.14 | 1.16 | 1.09 | 0.92 | 1.00 | 15.72 |
| | | LF | | polyamide | | | | | | | | | |
| A7 | Koch | SR2 | 350 | TFC | 1.380 | 1.64 | 0.86 | 1.37 | 1.41 | 1.63 | 0.93 | 0.99 | 37.34 |
| A8 | Saehan | NE70 | | TFC | 0.630 | 4.46 | 0.57 | 5.00 | 5.80 | 7.01 | 0.61 | 1.22 | 84.60 |
| A9 | Parker | NFA | 500 | TFC | 1.052 | 2.98 | 0.82 | 2.38 | 3.04 | 4.17 | 0.87 | 1.06 | 76.46 |
| A10 | DOW | NF270 | | TFC/Polyamide | 1.407 | 2.11 | 0.73 | 2.04 | 2.45 | 2.89 | 0.79 | 1.07 | 66.52 |
| A11 | GE | GM | 4K | TF | 0.593 | 1.11 | 0.73 | 1.21 | 1.22 | 1.16 | 0.81 | 1.01 | 18.72 |
| A12 | Alfa-Laval | ETNA01PP | 1K | Composite | 1.720 | 1.36 | 0.91 | 1.17 | 1.13 | 1.24 | 0.89 | 0.91 | 17.00 |
| | | | | fluoropolymer | | | | | | | | | |
| A13 | Alfa-Laval | GR95PP | 2K | PS PES | 0.258 | 1.38 | 0.70 | 1.37 | 1.45 | 1.51 | 0.84 | 1.10 | 33.69 |
| A14 | Sepro | PES-5 | 4K | PES | 4.267 | 1.15 | 0.92 | 1.11 | 1.13 | 1.13 | 0.97 | 0.99 | 11.04 |
| A16 | Parker | FAH | 4K | PVDF | 0.150 | 1.42 | 0.91 | 1.25 | 1.26 | 1.37 | 0.96 | 0.99 | 28.08 |
| A17 | GE | PT | 5K | PES | 2.328 | 1.22 | 0.82 | 1.19 | 1.21 | 1.24 | 0.92 | 1.05 | 17.85 |
| B1 | Nadir | NPD30 | | PES | 0.244 | 2.36 | 0.55 | 2.52 | 2.65 | 3.07 | 0.61 | 2.04 | 68.97 |
| B2 | Nadir | NPD30P | | PES | 0.213 | 2.31 | 0.52 | 2.58 | 2.71 | 3.06 | 0.60 | 2.10 | 69.07 |
| B3 | Alfa-Laval | NF99 | | TFC | 0.563 | 5.94 | 0.77 | 7.57 | 14.66 | 16.29 | 0.87 | 2.03 | 96.21 |
| B4 | Alfa-Laval | NF97 | | TFC | 0.370 | 1.02 | 0.60 | 1.41 | 1.51 | 1.26 | 0.14 | 1.73 | 96.63 |
| B5 | GE | GE | 1K | TF | 0.144 | 4.31 | 0.41 | 4.17 | 4.07 | 5.69 | 0.38 | 1.72 | 81.09 |
| B6 | Parker | ATF | 200 | TFC | 0.818 | 5.47 | 0.73 | 6.74 | 11.84 | 12.51 | 0.68 | 2.08 | 93.85 |
| B7 | GE | GH | 1K | TF | 0.366 | 2.32 | 0.54 | 2.26 | 2.06 | 2.81 | 0.54 | 1.72 | 61.17 |
| B8 | Sepro | NF3 | | TFC | 0.273 | 4.47 | 0.74 | 4.79 | 6.95 | 9.34 | 0.73 | 2.11 | 90.42 |
| B9 | Hydranautics | PVD1 | 250 | Polyvinyl alcohol | 1.061 | 3.32 | 0.60 | 3.88 | 4.87 | 5.66 | 0.56 | 1.87 | 82.35 |
| | | | | polyamine | | | | | | | | | |
| | | | | copolymer | | | | | | | | | |
| B10 | Hydranautics | ESNA3J | 130 | Composite | 0.630 | 3.64 | 0.79 | 4.21 | 8.40 | 10.90 | 0.76 | 2.11 | 92.73 |
| | | | | polyamide | | | | | | | | | |
| B13 | TriSep | TS80 | | TFC | 0.359 | 1.75 | 0.79 | 1.80 | 2.02 | 2.99 | 0.85 | 1.75 | 82.81 |
| B14 | TriSep | TS83 | | TFC | 0.342 | 1.70 | 0.77 | 1.78 | 2.31 | 2.83 | 0.82 | 1.79 | 82.98 |
| B15 | Hydranautics | 7470 | 500 | Sulfonated | 0.186 | 5.57 | 0.37 | 11.96 | 13.24 | 12.07 | 0.44 | 2.63 | 92.86 |
| | | | | polyethersulfone | | | | | | | | | |
| B16 | DOW | NF90 | | TFC/Polyamide | 0.507 | 1.26 | 0.75 | 1.51 | 1.31 | 1.66 | 0.78 | 1.73 | 89.89 |
| B17 | Hydranautics | 7450 | 550 | Sulfonated | 0.653 | 3.60 | 0.28 | 5.51 | 5.11 | 6.30 | 0.37 | 2.41 | 83.50 |
| | | | | polyethersulfone | | | | | | | | | |
| B20 | Hydranautics | ESNA1- | 190 | Composite | 2.584 | 1.85 | 0.59 | 2.32 | 2.44 | 2.74 | 0.82 | 1.72 | 63.07 |
| | | LF2 | | polyamide | | | | | | | | | |

Table lb. Membrane Performance at 30° C.

| | | | | | Water | | | | | | | | |
|---|---|---|---|---|---|---|---|---|---|---|---|---|---|
| | | | | | Flux | alpha (30C) | | | | | | | |
| Test ID | Manufacturer | Membrane | MWCO | Type/Material | (LMH/psi) | I3P | MP | Trp | Ala | Pyr | HMO | HMG | Rej % |
| A3 | Sepro | NF2 | | TFC | 1.395 | 2.98 | 0.61 | 3.07 | 3.22 | 3.97 | 0.63 | 0.92 | 58.94 |
| B11 | Sepro | NF5 | | TFC | 0.976 | 42.65 | 0.63 | 16.16 | | 62.47 | 0.50 | | 98.33 |
| B18 | GE | HL | 150~300 | TF | 0.880 | 7.67 | 0.63 | 3.87 | | 10.72 | 0.52 | | 90.23 |
| B19 | GE | DL | 150~300 | TF | 0.459 | 33.22 | 0.66 | 15.35 | | 54.81 | 0.43 | | 97.63 |
| A1 | Koch | SR3 | 200 | TFC | 0.766 | 1.47 | 0.82 | 1.32 | 1.39 | 1.69 | 0.88 | 0.96 | 39.85 |
| A2 | DOW | NF | | TFC/ | 0.445 | 3.86 | 0.71 | 3.85 | 4.31 | 5.52 | 0.74 | 1.04 | 65.33 |
| | | | | Polypiperazine | | | | | | | | | |
| | | | | amide | | | | | | | | | |
| A5 | Hydranautics | ESNA1-LF | 270 | Composite | 5.142 | 1.11 | 0.87 | 1.18 | 1.15 | 1.15 | 0.91 | 0.96 | 10.96 |
| | | | | polyamide | | | | | | | | | |
| A7 | Koch | SR2 | 350 | TFC | 1.380 | 1.91 | 0.82 | 1.64 | 1.56 | 1.95 | 0.83 | 0.91 | 37.12 |
| A8 | Saehan | NE70 | | TFC | 0.630 | 5.12 | 0.42 | 5.31 | 5.32 | 6.90 | 0.42 | 0.98 | 65.71 |
| A9 | Parker | NFA | 500 | TFC | 1.052 | 5.99 | 0.65 | 4.57 | 5.11 | 7.51 | 0.66 | 0.97 | 68.11 |
| A10 | DOW | NF270 | | TFC/Polyamide | 1.407 | 3.01 | 0.66 | 2.75 | 2.88 | 3.77 | 0.67 | 0.92 | 56.44 |
| A11 | GE | GM | 4K | TF | 0.593 | 1.27 | 0.87 | 1.21 | 1.27 | 1.31 | 0.93 | 1.04 | 19.60 |
| A12 | Alfa-Laval | ETNA01PP | 1K | Composite | 1.720 | 1.42 | 0.86 | 1.22 | 1.13 | 1.26 | 0.73 | 0.86 | 12.74 |
| | | | | fluoropolymer | | | | | | | | | |
| A13 | Alfa-Laval | GR95PP | 2K | PS PES | 0.258 | 1.44 | 0.70 | 1.39 | 1.30 | 1.50 | 0.76 | 0.92 | 26.55 |
| A14 | Sepro | PES-5 | 4K | PES | 4.267 | 1.36 | 1.06 | 1.14 | 1.24 | 1.31 | 1.09 | 1.14 | 17.39 |
| A16 | Parker | FAH | 4K | PVDF | 0.150 | 1.54 | 0.99 | 1.25 | 1.24 | 1.40 | 0.98 | 1.00 | 25.06 |
| A17 | GE | PT | 5K | PES | 2.328 | 1.19 | 0.85 | 1.20 | 1.17 | 1.24 | 0.92 | 1.02 | 15.46 |
| B1 | Nadir | NPD30 | | PES | 0.244 | 2.34 | 0.54 | 2.51 | | 2.68 | 0.53 | | 66.79 |
| B2 | Nadir | NPD30P | | PES | 0.213 | 2.31 | 0.53 | 2.55 | | 2.68 | 0.54 | | 65.94 |
| B3 | Alfa-Laval | NF99 | | TFC | 0.563 | 14.29 | 0.48 | 19.32 | | 29.83 | 0.29 | | 97.33 |
| B4 | Alfa-Laval | NF97 | | TFC | 0.370 | 1.48 | 0.61 | 1.91 | | 2.22 | 0.33 | | 96.81 |
| B5 | GE | GE | 1K | TF | 0.144 | 3.47 | 0.42 | 3.38 | | 3.85 | 0.32 | | 74.30 |
| B6 | Parker | ATF | 200 | TFC | 0.818 | 11.67 | 0.52 | 14.56 | | 21.39 | 0.37 | | 95.85 |
| B7 | GE | GH | 1K | TF | 0.366 | 1.98 | 0.55 | 2.03 | | 2.13 | 0.49 | | 54.60 |
| B8 | Sepro | NF3 | | TFC | 0.273 | 9.16 | 0.55 | 10.36 | | 16.06 | 0.43 | | 94.28 |
| B9 | Hydranautics | PVD1 | 250 | Polyvinyl alcohol | 1.061 | 3.50 | 0.51 | 4.35 | | 5.20 | 0.43 | | 81.28 |
| | | | | polyamine | | | | | | | | | |
| | | | | copolymer | | | | | | | | | |
| B10 | Hydranautics | ESNA3J | 130 | Composite | 0.630 | 8.93 | 0.65 | 10.79 | | 22.31 | 0.94 | | 96.11 |
| | | | | polyamide | | | | | | | | | |
| B13 | TriSep | TS80 | | TFC | 0.359 | 2.09 | 0.68 | 2.71 | | 4.16 | 0.58 | | 91.05 |
| B14 | TriSep | TS83 | | TFC | 0.342 | 2.07 | 0.65 | 2.75 | | 4.00 | 0.54 | | 92.03 |
| B15 | Hydranautics | 7470 | 500 | Sulfonated | 0.186 | 6.18 | 0.22 | 12.29 | | 11.57 | 0.21 | | 92.15 |
| | | | | polyethersulfone | | | | | | | | | |
| B16 | DOW | NF90 | | TFC/Polyamide | 0.507 | | | | | | | | 88.89 |
| B17 | Hydranautics | 7450 | 550 | Sulfonated | 0.653 | 3.31 | 0.25 | 4.63 | | 4.78 | 0.27 | | 78.21 |
| | | | | polyethersulfone | | | | | | | | | |
| B20 | Hydranautics | ESNA1-LF2 | 190 | Composite | 2.584 | 1.83 | 0.53 | 2.34 | | 2.66 | 0.63 | | 62.58 |
| | | | | polyamide | | | | | | | | | |

Thus, in addition to concentrating the feed stream, certain membranes further purified the mixture. Upon further analysis of the specific surface chemistry of the membranes to understand this observation, it was discovered that use of membranes having characteristics such as zeta potential of from about −19.0 to −6.0 result in alpha (α) values greater than 30 for I3P. Zeta potential is generally the electric potential that exists across the interface of liquids and solids. It is also known as electro-kinetic potential. Details regarding the protocol used for measuring zeta potential for the membranes identified as Test ID A3, B11, B18 and B19 are provided later in this document. It was also observed with certain membranes, particularly those having a zeta potential of from about −19.0 to −6.0 that monatin was rejected (retained) at values greater than 95%, greater than 96%, greater than 97% and in some instances greater than 98%. The zeta potentials for the membranes identified as Test ID A3, B11, B18 and B19 are provided in Table 2.

TABLE 2
Zeta Potential

| | | | Zeta |
|---|---|---|---|
| Test ID | Manufacturer | Membrane | potential (mV) |
| A3 | Sepro | NF2 | −63.73 |
| B11 | Sepro | NF5 | −6.70 |
| B18 | GE | HL | −32.45 |
| B19 | GE | DL | −18.40 |

Accordingly, in one embodiment is a method of recovering monatin from a mixture including monatin and at least one of I3P, tryptophan or pyruvate

Based in part on the performance data in Tables 1a-b and Table 2, a suitable membrane for removing water in the permeate, retaining monatin in the retentate and also removing I3P in the permeate is a DL Nanofiltration membrane from GE Osmonics (Hopkins, MN). Membranes of this type include the DL1812 and DL 2540 spiral wound membranes where the GE DL1812 membrane has a diameter of about 4.6 cm, a length of about 30.5 cm, and surface area of about 0.25 m² and where the GE DL2540 membrane has a diameter of about 6.1 cm, a length of about 101.6 cm, and surface area of about 1.7 m². An alternative suitable membrane is the GE Osmonics DESAL DL3840C-30D spiral wound membrane having a diameter of about 9.6 cm, length of about 98.4 cm and surface area of about 7.8 m². In another aspect, a suitable membrane is a Sepro NF5 Nanofiltration membrane available from Sepro (Atlanta, Ga.).

In another embodiment, other non-monatin components such as tryptophan, pyruvate and alanine are also partially removed by a membrane. Table 3 lists average percent removal of non-monatin components at various temperatures using a GE Osmonics DL membrane having a zeta potential of from about −19.0 to −6.0:

TABLE 3
Average Percent Removal

| | Molecular | % Removal | % Removal |
|---|---|---|---|
| Component | Mass | at 20-35° C. | at 15° C. |
| Monatin | 314 | 0 | 0 |
| Monatin | 313 | 0 | 5 |
| Precursor | | | |
| I3P | 225 | 50 | 20 |
| Tryptophan | 204 | 30 | 0 |
| HMG | 199 | 0 | 0 |
| HMO | 198 | 5 | 0 |
| Pyruvate | 110 | 80 | 60 |
| Alanine | 89 | 50 | 25 |

As can be seen in Table 3, the membrane clearly and surprisingly separates monatin from other components having a similar molecular mass.

In another embodiment, diafiltration may be used in conjunction with a membrane operation. The retentate is washed with fresh water to further remove impurities.

Aspects of the invention are illustrated in the following non-limiting examples.

### EXAMPLES

### Example 1

### Zeta Potential Measurement

The Zeta potentials of membrane samples A3, B11, B18 and B19 from Tables 1a-b were measured using a Beckman Coulters DelsaNano C particle and Zeta potential analyzer. The DelsaNano utilizes Photon Correlation Spectroscopy and Electro-phoretic Light Scattering techniques to determine particle size and zeta potential of materials. Electrophoretic light scattering is the method most popularly used to determine the velocity of the particles suspended in a liquid medium under an applied electric field. In order to determine the speed of the particles movement, the particles are irradiated with a laser light and the scattered light emitted from the particles is detected. Because the frequency of the scattered light is shifted from the incident light in proportion to the speed of the particles movement, the electrophoretic mobility of the particles can be measured from the frequency shift of the scattered light. When an electric field is applied to charged particles in the suspension, particles move toward an electrode opposite to their surface charge. Because the velocity is proportional to the amount of charge of the particles, zeta potential can be estimated by measuring the velocity of the particles.

In an electrophoresis cell consisting of membrane and quartz cells, asymmetric electroosmotic flow occurs due to the accumulation of ions on the membrane surface during the electrophoresis method. The electrophoretic flow of a standard particle then takes place due to the induced electroosmotic flow. The electrophoretic mobility of the particle can be measured, and exhibits a parabolic flow velocity profile. From determined electrophoretic mobility, the Zeta potential can be calculated. Polystyrene latex particles with a diameter 520 nm coated with hydroxypropyl cellulose (HPC) and a molecular weight of 300,000 g/mol were used as mobility-monitoring particles. Zeta potential measurements were conducted with membrane specimens in a background aqueous solution of pH 8.

TABLE 4
Zeta potentials of the membrane specimens

| | | | | B18 | | | |
|---|---|---|---|---|---|---|---|
| | | | | Test | Test | Test | |
| | | | | run | run | run | Aver- |
| Specimen | B11 | A3 | B19 | 1 | 2 | 3 | age |
| Zeta | −6.70 | −63.73 | −18.46 | −31.04 | −26.80 | −39.50 | −32.44 |
| potential | | | | | | | |
| (mV) | | | | | | | |

### Example 2

### Nanofiltration Process Description

The following information is provided as an example of the process of configuring, cleaning and operating the membrane. Additional information is included regarding sampling and analysis procedures for a particular test set-up using a membrane of 1.7 m².

Table 5 shows the detailed results for the 8 components in the feed, permeate and retentate streams from the nanofiltration test performed at 17° C. on the UF permeate from Trial FW-161E1-N

21.34 kg feed liquor ((S0) i.e. the permeate from the ultrafiltration step) was treated on a GE Osmonics DL2540 NF membrane to a concentration factor of 6.5. After 129 minutes the permeate (S9) and the retentate (S10) were drained, weighted and analyzed. Subsequently, 3.02 kg water was recirculated in the membrane during 15 minutes to recover the components from the void volume of the membrane (the feed spacers and membrane surface area) and possibly adsorbed by the membrane. After 15 minutes, the liquor with the “extracted” remaining components (S11) was drained, weighted and analyzed. This rinse (S11) was and combined with the retentate (S10) into the total recovered fraction (S12). The overall concentration factor of this combined fraction is 3.35.

Subsequently, another 11.94 kg of water was recirculated in the membrane during 15 minutes to check if more components from the void volume of the membrane and possibly adsorbed by the membrane can be recovered. After 15 minutes, the liquor with the “extra extracted” remaining components (S13) was drained, weighted and analyzed.

Thereafter, another 6.08 kg of water was recirculated in the membrane during 15 minutes to check if more components from the void volume of the membrane and possibly adsorbed by the membrane can be recovered. After 15 minutes, the liquor with the “extra extracted” remaining components (S14) was drained, weighted and analyzed.

TABLE 5
Detailed results for the 8 components in the feed, peuneate and retentate streams
(FW161E1-N)

| | | Total | Total | | | | | |
|---|---|---|---|---|---|---|---|---|
| | Density | weight | volume | | | | | |
| | (g/ml) | (kg) | (l) | Comments | | | | |
| Feed (S0) | 1.000 | 21.34 | 21.34 | | | | | |
| Permeate (S9) | 1.000 | 17.42 | 17.42 | | | | | |
| Retentate (S10) | 1.085 | 3.58 | 3.30 | Concentration factor: 6.47 | | | | |
| Recovery rinse (S11) | 1.000 | 3.02 | 3.02 | | | | | |
| Total recovered fraction (S12) | 1.036 | 6.60 | 6.37 | Overall concentration factor: 3.35 | | | | |
| Extra Rinse 1 (S13) | 1.000 | 11.94 | 11.94 | | | | | |
| Extra Rinse 2 (S14) | 1.000 | 6.08 | 6.08 | | | | | |
| | Concentration (mM) | | | | | | | |
| | Mon | I3P | MP | Trp | Ala | Pyr | HMO | HMG |
| Feed (S0) | 25.87 | 48.37 | 24.90 | 36.27 | 49.27 | 48.07 | 15.50 | 2.77 |
| Permeate (S9) | 0.37 | 12.73 | 0.00 | 3.10 | 14.85 | 35.83 | 0.00 | 0.00 |
| Retentate (S10) | 148.00 | 217.60 | 142.60 | 211.73 | 202.33 | 112.23 | 87.60 | 13.70 |
| Recovery rinse (S11) | 18.67 | 40.20 | 20.93 | 28.00 | 39.23 | 34.83 | 11.73 | 2.17 |
| Total recovered fraction (S12) | 79.60 | 132.03 | 85.00 | 112.17 | 134.40 | 74.70 | 54.90 | 9.80 |
| Extra Rinse 1 (S13) | 1.03 | 2.07 | 1.23 | 1.53 | 2.17 | 1.67 | 0.57 | 0.10 |
| Extra Rinse 2 (S14) | 0.80 | 1.50 | 1.00 | 1.20 | 1.60 | 1.20 | 0.50 | 0.10 |
| | total quantity (mmol) | | | | | | | |
| | Mon | I3P | MP | Trp | Ala | Pyr | HMO | HMG |
| Feed (S0) | 552.0 | 1032.1 | 531.4 | 773.9 | 1051.4 | 1025.7 | 330.8 | 59.0 |
| Permeate (S9) | 6.4 | 221.8 | 0.0 | 54.0 | 258.7 | 624.2 | 0.0 | 0.0 |
| Retentate (S10) | 488.3 | 718.0 | 470.5 | 698.6 | 667.6 | 370.3 | 289.0 | 45.2 |
| Recovery rinse (S11) | 56.4 | 121.4 | 63.2 | 84.6 | 118.5 | 105.2 | 35.4 | 6.5 |
| Total recovered fraction (S12) | 507.1 | 841.1 | 541.5 | 714.6 | 856.2 | 475.9 | 349.7 | 62.4 |
| Extra Rinse 1 (S13) | 12.3 | 24.7 | 14.7 | 18.3 | 25.9 | 19.9 | 6.8 | 1.2 |
| Extra Rinse 2 (S14) | 4.9 | 9.1 | 6.1 | 7.3 | 9.7 | 7.3 | 3.0 | 0.6 |
| “Losses” (S15) | 17.2 | 33.8 | 20.8 | 25.6 | 35.6 | 27.2 | 9.8 | 1.8 |
| | Total relative quantity recovered (%) | | | | | | | |
| Feed (S0) | 100.0 | 100.0 | 100.0 | 100.0 | 100.0 | 100.0 | 100.0 | 100.0 |
| Permeate (S9) | 1.2 | 21.5 | 0.0 | 7.0 | 24.6 | 60.9 | 0.0 | 0.0 |
| Retentate (S10) | 88.5 | 69.6 | 88.5 | 90.3 | 63.5 | 36.1 | 87.4 | 76.6 |
| Rinse (S11) | 10.2 | 11.8 | 11.9 | 10.9 | 11.3 | 10.3 | 10.7 | 11.1 |
| Total recovered fraction (S12) | 91.9 | 81.5 | 101.9 | 92.3 | 81.4 | 46.4 | 105.7 | 105.7 |
| Extra Rinse 1 (S13) | 2.2 | 2.4 | 2.8 | 2.4 | 2.5 | 1.9 | 2.0 | 2.0 |
| Extra Rinse 2 (S14) | 0.9 | 0.9 | 1.1 | 0.9 | 0.9 | 0.7 | 0.9 | 1.0 |
| “Losses” (S15) | 3.1 | 3.3 | 3.9 | 3.3 | 3.4 | 2.7 | 3.0 | 3.1 |
| Total mass balance recovery | 103.0 | 106.1 | 104.4 | 111.5 | 102.8 | 109.9 | 101.1 | 90.7 |

Total recovered fraction (S12) = Retentate (S10) + Recovery Rinse (S11)
“Losses” (S15) = Extra rinse 1 (S13) + Extra Rinse 2 (S14)

### Example 3

### Nanofiltration Using DL Membrane at 17 C with Diafiltrations

24 kg feed liquor ((S0) i.e. the permeate from the ultrafiltration step) was treated on a GE Osmonics DL2540 NF membrane to a concentration factor of 2.5×. After that, diafiltration water was added to the feed tank in order to maintain a constant volume. A diafiltration equivalent would be the volume of the retentate at a 2.5× concentration factor. In this case, it is approximately equal to 9.6 L. Samples were taken after each diafiltration equivalent volume was permeated from the retentate. A total of 10 diafiltrations were performed. After the 10^{th} diafiltration equivalent volume, the retentate was concentrated further to a 4.4× factor based on the original UF permeate volume. At completion, the permeate (S33) and the retentate (S32) were drained, weighted and analyzed. Subsequently, 9.9 kg water was recirculated in the membrane during 15 minutes to recover the components from the void volume of the membrane and possibly adsorbed by the membrane. After 15 minutes, the liquor with the “extracted” remaining components (S34) was drained, weighted and analyzed. The overall concentration factor of this combined fraction is 4.3×.

Subsequently, another 6.08 kg of water was pumped through the membrane to check if more components from the void volume of the membrane and adsorbed by the membrane can be recovered. After 15 minutes, the liquor with the “extra extracted” remaining components (S35) was drained, weighted and analyzed. Thereafter, another 11.94 kg of water was recirculated in the membrane during 15 minutes to check if more components from the void volume of the membrane and possibly adsorbed by the membrane can be recovered. After 15 minutes, the liquor with the “extra extracted” remaining components (S36) was drained, weighted and analyzed. Table 6 shows the detailed results for the 8 components in the feed, permeate and retentate from Trial FW-242E1-N.

TABLE 6a
Concentrations for the Diafiltration Runs for Example FW-242-E1-N, DL
Membrane at a Feed Pressure of 500 PSI (through 10 diafiltrations)

| | Monatin | Trp | Pyruvate | I3P | MP | HMO | Ala. | HMG |
|---|---|---|---|---|---|---|---|---|
| Feed | 504.75 | 988.18 | 1260.99 | 1157.91 | 512.75 | 403.45 | 815.78 | 70.20 |
| retentate 2.5× | 408.84 | 790.27 | 1078.02 | 1029.58 | 472.25 | 403.10 | 667.91 | 54.81 |
| retentate at 1D | 418.73 | 778.73 | 835.53 | 919.27 | 449.53 | 386.00 | 642.05 | 57.76 |
| retentate at 2D | 442.84 | 809.47 | 666.68 | 875.08 | 438.99 | 375.31 | 618.44 | 60.78 |
| retentate at 3D | 418.87 | 766.01 | 525.97 | 797.57 | 421.73 | 360.53 | 525.02 | 56.42 |
| retentate at 4D | 444.65 | 788.80 | 438.01 | 763.20 | 424.74 | 365.01 | 511.96 | 60.68 |
| retentate at 5D | 419.39 | 743.98 | 412.69 | 811.01 | 475.88 | 409.81 | 439.50 | 56.49 |
| retentate at 6D | 427.86 | 741.94 | 327.25 | 758.87 | 466.42 | 403.41 | 405.30 | 57.36 |
| retentate at 7D | 404.16 | 680.49 | 264.11 | 699.29 | 451.16 | 386.30 | 349.65 | 53.57 |
| retentate at 8D | 391.39 | 656.13 | 200.93 | 611.37 | 411.39 | 354.25 | 311.40 | 51.42 |
| retentate at 9D | 446.20 | 716.07 | 176.34 | 602.08 | 423.79 | 364.37 | 335.14 | 60.40 |
| retentate at 10D | 434.59 | 682.24 | 146.47 | 557.94 | 415.32 | 357.50 | 299.69 | 58.78 |
| Final retentate | 415.13 | 644.18 | 128.68 | 500.48 | 385.68 | 334.47 | 292.45 | 56.46 |
| at 4.3× | | | | | | | | |
| millimoles in | 63.02 | 94.03 | 20.46 | 79.85 | 55.76 | 47.18 | 42.56 | 8.91 |
| recirc | | | | | | | | |

Where all data in Table 6a is expressed in units of millimoles.

TABLE 6b
Alpha values for Example FW-242-E1-N, DL Membrane at a
Feed Pressure of 500 PSI (through 10 diafiltrations)

| DIAFILTRATION | ALPHA | ALPHA | ALPHA | ALPHA |
|---|---|---|---|---|
| RUN | I3P | TRYP | PYRUVATE | ALANINE |
| 2.5X | 18.40 | 8.28 | 65.86 | 28.28 |
| 1D | 13.44 | 5.65 | 48.36 | 19.57 |
| 2D | 14.42 | 6.02 | 51.48 | 21.48 |
| 3D | 26.78 | 11.48 | 96.36 | 40.69 |
| 4D | 26.80 | 11.84 | 96.44 | 41.69 |
| 5D | 23.27 | 10.71 | 80.28 | 36.26 |
| 6D | 23.12 | 9.80 | 78.45 | 33.78 |
| 7D | 21.38 | 9.50 | 73.45 | 33.52 |
| 8D | 21.13 | 9.54 | 72.07 | 35.19 |
| 9D | 23.72 | 9.97 | 78.44 | 33.28 |
| 10D | 23.37 | 9.56 | 74.18 | 37.70 |
| 4.3X | 17.00 | 6.01 | 140.87 | 33.83 |

### Example 4

### Operation of a Nanofiltration Process

THE EQUIPMENT: The nano-filtration process uses a DL spiral wound membrane (diameter 6.08 cm, length 101.60 cm, SA and feed spacer) to accomplish the permeate concentration. The membrane is inserted into a stainless steel housing (diameter 6.35 cm, length 115.25 cm). The positive displacement pump (1.5 horse power) is set at 40 Hz to maintain an average flow rate of 2.1 GPM (gallons per minute, equivalent to 7.95 LPM (liters per minute)) through the housing. Pressure is measured using glycerin-filled psi gauges located on the inlet and on the retentate port of the housing. The pressure is adjusted using a needle valve on the retentate line. The pressure inside the housing is maintained at a feed pressure of 300 (20.68 bar) or 500 (34.47 bar) psi throughout the run. The two different pressure constants used for concentration are correlated to the initial permeate flux. To determine the pressure to be used during the run; the nanofiltration is initially started at a feed pressure of 300 psi. If the permeate flux on the flow meter is above 200 mL/min upon beginning the run, then the entire run is maintained at a feed pressure of 300 psi. If the permeate flux is lower than 200 mL/min at the start of the run, the pressure is increased to a feed pressure of 500 psi for the duration of the run. This pressure increase is done so that the entire nanofiltration can be completed in a nine hour time period. Temperature is monitored throughout the run using a digital stainless steel thermometer attached to the housing. As the run progresses temperature increases from friction occurring on the membrane. The thermometer is used to monitor that the housing temperature never reaches 50 degrees Celsius, which may damage the membranes. Lower temperature is also preferred for preventing degradation of monatin or reaction intermediates. When the temperature reaches 40 degrees Celsius the membrane, cooling is applied to maintain the desired temperature.

Membrane Preparation and Cleaning: Between runs, the membranes must be cleaned and prepared for continued use. Due to the specific nature of different membranes, the membrane should be cleaned and stored according to the instructions of the manufacturer and/or those of the vendors of cleaning materials. One skilled in the art might modify these instructions for greater efficiency without risking the membranes performance.

Initiating a Membrane Run: The retentate line is a stainless steel braided hose (diameter ¼ in, length 0.61 m) and the permeate line is a stainless steel braided hose (diameter ¼ in, length 0.61 m) connected to Norprene food grade tubing (diameter ¼ in, thickness 24 mm, length 0.9 m). Norprene tubing was chosen because it is impermeable to oxygen. The juncture between the braided hose and Norprene tubing on the permeate line has a digital paddle wheel flow meter calibrated for 100-1000 mL/min. There is also a sampling port located 1 ft (0.3 m) from the flow meter. This port is made with a plastic ball valve (diameter ¼ in). The port is necessary so samples of permeate can be taken during the run without exposing the permeate tanks contents to oxygen. After the pH has stabilized and the samples are taken, the pump is shut off. The intake line on the pump, a braided stainless steel hose (diameter %, length 1.22 m) is then connected to a nitrogen gas line with a stainless steel quick connect (diameter ⅜ in). The gas line has nitrogen flowing through at 0.16 SCFM (4.72 LPM). After connecting, the needle valve on the retentate line is turned until it is completely open and then the pump is started at normal operating speed (40 Hz) for 5 min. Then the pump speed is decreased to 5 Hz and the needle valve is turned clockwise until it is within one turn of being entirely shut. By closing the valve backpressure is created, blowing the remaining water out of the permeate tube. The membrane is left under these conditions for 55 minutes to remove all remaining water in the housing and insure that all oxygen has been evacuated. While the membrane has nitrogen pumping through it, tare weights of both the concentrate tank (retentate) and permeate tank are recorded using a floor scale (max weight capacity 220 kg). These 16-gallon (60.55 L) plastic tanks are sparged with nitrogen for 30 minutes at 0.33 SCFM (9.44 LPM). This is accomplished with food grade Norprene tubing (diameter ¼, thickness 17 mm) that is connected to a barbed nipple (diameter ¼ in) that feeds into the top of the tank. A piece of Norprene tubing is attached to the barbed nipple on the inside of the tank to direct the nitrogen flow to the bottom. This insures nitrogen is flushed throughout the tank and is not just filling the headspace. The UF permeate is collected in a 25L carboy under nitrogen and transferred under nitrogen into the retentate tank using a peristaltic pump (horse power 0.1). To accomplish this Norprene tubing (diameter % in, thickness 24 mm, length 5 ft) is attached to acetyl quick connects (diameter ¼ in) located on the pump, creating transfer lines. Before transfer the transfer lines have de-gassed water (pH 7.5) pumped through them, followed by nitrogen to clear the lines of any contaminants and oxygen. The 25 L carboy is connected to the feed transfer line via the acetyl quick connect. The retentate tank is tared at this point so that the UF permeate weight can be measured. The outlet transfer line from the pump is then connected to an acetyl quick connect on the retentate tank. This line is set up in the same manner as the nitrogen line directing the UF permeate to the base of the tank. After all lines are connected the Humboldt clamp (size 2.5 cm) located on the outlet line of the carboy is opened to allow flow through the feed line of the pump. The peristaltic pump is turned on and run at a setting of 9 RPMs. Once the entire contents have been pumped into the tank the UF permeate weight is recorded. The tank is then lifted off the scale. The scale is zeroed and the tank is placed back on, the weight of the tank with the loaded permeate is then recorded. At this point the membrane is completely sparged. The pump is shut off and the nitrogen line is disconnected from the feed line on the pump. The feed line is connected to a stainless steel quick connect plug (diameter ⅜ in) on the base of the concentrate tank and the retentate line is connected to a stainless steel quick connect plug (diameter % in) on the top of the tank. The permeate line is connected to the second tank with an acetyl quick connect coupling (diameter % in); this tank gathers all the contents permeated through the membrane. The initial weight of the tank, permeate loaded and attached lines is recorded. These values are used to calculate the concentration factors (2×, 4×, 6×) along with the known holdup volume of the membrane housing (2 L) using this formula (total weight−((permeate weight−(permeate weight/concentration factor))−holdup volume)). A S0 sample is taken from the retentate tank before the run is started. After the samples are completed the conductivity and pH is measured and recorded from the original 50 mL tube. At this point the pump is turned on and allowed to ramp up to 40 Hz. The pump is allowed to run for approximately two minutes without any backpressure. This provides enough time for the concentrate tank liquid to force all of the nitrogen gas out of the housing, eliminating the chance of cavitation once backpressure is added. The needle valve is slowly turned clockwise until 300 psi of backpressure is added. After a minute has passed the permeate flux is measured by checking the flow meter on the permeate line. At this point using the protocol listed earlier, the backpressure that the run will be operated at is decided on (300 or 500 psi). When the concentrate tank is 10 kg away from the 2× concentration value an inline permeate sample is taken using the sampling port. First, values of time, temp, permeate flux, feed flow rate, inlet pressure, retentate pressure, pump setting and weight of concentrate are recorded. Then a 25 to 30 mL sample is taken in a tared 50 ml plastic cylindrical tube from the sampling port. The sample is weighed. The conductivity of the undiluted 50 mL sample is measured and recorded. When the concentrate tank is 5 kg from the 2× concentration value a second inline permeate sample is taken and follows the same protocol for data collection, sampling and dilutions described above. The next samples occur at the 2× concentration value. First, data is taken for the same operating variables as earlier (time, temp, etc). Then an inline sample is collected from the permeate sampling port. After this a second sample is taken from the concentrate tank. Both samples are collected in the pre-tared plastic tubes. The concentrate tank is stirred before sampling using a clean plastic spatula. The sample is then collected in the same manner as the S0 sample. After the dilutions are complete the conductivity of the undiluted concentrate and inline undiluted permeate samples are measured and recorded. The next sampling point is at the 4× concentration value. The protocol for data collection, sampling and dilutions follows the same format as the 2× sample stated above. Just prior to shutting the pump off a 6× concentration sample is taken. This sample differs from the 2× and 4× samples only in how the permeate flux is measured. The permeate flux at this point is usually too slow for the digital paddle wheel flow meter to read. To measure this drop off in flux an approximation is done by timing the flow rate of permeate from the inline sample port into a graduated cylinder for 30 seconds using a stopwatch. When the weight of the concentrate tank is 1 kg less than the 6× concentration value the pump is turned off. The intake line on the pump is hooked up to the nitrogen gas line as described earlier and the entire contents of the housing are blown into the concentrate tank. To insure all liquid is evacuated the housing is tilted at a 45 degree angle by lifting the inlet side into the air. The pump is then shut down and unhooked from the nitrogen line. Samples from the concentrate tank and the permeate tank are collected. Sampling and dilutions from the concentrate tank follow the protocol described earlier. The permeate sample is a composite sample; instead of taking it from the port it is collected from the tank. A 3 foot plastic spatula is used to stir the tank and then using a 10 mL transfer pipette 25 to 30 mL of permeate are collected in a pre-tared plastic tube. After the dilutions have been made the undiluted concentrate and composite permeate samples are measured for conductivity. The composite permeate sample is also measured for pH. The inlet line on the pump is then attached to a nitrogen sparged 16-gallon tank using a stainless steel quick connect plug (diameter % in) at the base of the tank. The retentate line is attached to the top of the tank with a stainless steel quick connect plug (diameter % in). On the inside of the tank is a 3 foot (0.9 m) piece of Norprene tubing (thickness 24 mm) attached to the retentate quick disconnect plug. The purpose of the tubing is to direct the retentate flow to the base of the tank in order to keep a consistent feed for the pump. The permeate line is connected to an acetyl quick connect coupling (diameter ¼ in) on top of the tank. This connection also has a line attached on the inside of the tank for the same purpose as the line directing the retentate. The 16-gallon tank has 3 L of pH 7.5 de-gassed water pumped into it. The membrane pump is turned on and allowed to ramp up to 40 Hz and then run for 2 minutes without backpressure to expel all of the nitrogen gas inside of the housing. The needle valve is then slowly closed adding backpressure to the specifications of the run (300 or 500 psi). The 3 L de-gassed water is re-circulated for 15 minutes. Then the pump is shut off. The intake line on the pump is then hooked up to the nitrogen line in the same manner as before and all of the housing's contents are blown back into the 16-gallon tank. A sample is taken from this mixture using the same protocol as earlier. The dilutions for this sample are the same as the dilutions made for all samples from the concentrate tank. After the dilutions are completed the conductivity is measured. After the sampling, a portion of the mixture from the recirculation tank is pumped into the retentate tank. The exact mass added depends on how much is required to bring the concentrate tank to exactly a 6× concentration. After adding back a portion of the re-circulated water, the concentrate tank has a final sample taken from it once it has been mixed. The dilutions are the same as earlier and when they are completed the conductivity and pH of the undiluted sample is measured. The concentrate tank has had all of the samples taken at this point and is ready to load onto the DAC column. The concentrate is usually left in the tank with a constant nitrogen supply flowing in. This eliminates the chance for oxygen degradation until the DAC chromatography runs can be started.

Completing a Membrane Run: At this point the membrane needs to be cleaned to remove any residual contents from the run. First a tared 5-gallon pail has 12 kg of de-ionized water pumped into it. Then the feed and retentate lines are connected to stainless steel quick connect plugs (diameter % in) while the permeate line is connected to an acetyl quick connect socket (diameter ¼ in). All of the lines are placed directly into the pail and the pump is turned on and allowed to run for two minutes in order to force all of the nitrogen out of the housing. Backpressure is then slowly added until 300 psi is reached. After 15 minutes of recirculation the backpressure is released and the pump is shut down. The pail is sampled after thorough mixing using the protocol described earlier. The dilutions made from this sample follow the protocol listed earlier for permeate samples, except that the Alanine-HMG sample is not diluted. Instead 1 undiluted mL is placed into a vial for analysis. This dilution change is due to the low concentration of these two components left on the membrane after the run. The undiluted sample is then measured for conductivity. The Intake line on the pump is now placed into a pail containing 6 kg of de-ionized water, while the retentate and permeate lines are placed into an empty 5-gallon pail. The pump is turned on and then 300 psi of backpressure is added. The pump is then shut down as the last of the water is pulled into the pump; there is no re-circulation of this water. The pail with the retentate and permeate lines now contains the 6 kg of water. This pail is sampled, has dilutions made and measured for conductivity using the same protocol as the 12 kg water recirculation. The rest of the cleaning follows the cleaning steps described earlier except for one change. The first water rinse is omitted because pure de-ionized water has already been sent through the housing with the 12 kg and 6 kg pails. The first step in the cleaning is the last sample that is collected for analysis. The sample is diluted and measured for conductivity in the same manner as the two water rinses before it. The final three samples of the run collected during the cleaning are used to determine loss of product, intermediates and reaction components. This amount of reaction material is the residual hold up volume of the membrane and cannot be recovered even after forcing nitrogen gas into the housing. After the cleaning is complete, the membrane may have alkaline water (deionized water adjusted to pH 10) pumped into the housing. This is to store the membrane if it will not be used within a 48 hours.

### Example 5

### Nanofiltration Using NF-5 Membrane at 15° C. with Diafiltrations

The description of sample creation follows Table 7.

TABLE 7
Detailed results for the 8 componentsin the feed, permeate and retentate streams
(FW-242E14-N)

| | | Total | Total |
|---|---|---|---|
| | Density | Weight | volume |
| | (g/mL) | (kg) | (l) |
| Feed S9 | 1.01 | 26.80 | 26.41 |
| Permeate S33 | 1.00 | 97.80 | 97.97 |
| Retentate S32 | 1.04 | 5.52 | 5.28 |
| Recovery Rinse | 1.00 | 10.00 | 10.02 |
| S34 | | | |
| Total Recovered | 1.02 | 15.52 | 15.19 |
| Fraction | | | |
| [S34 + S32] | | | |
| Extra Rinse 1 S35 | 1.00 | 6.02 | 6.04 |
| Extra Rinse 2 S36 | 1.00 | 12.02 | 12.05 |

| | Concentration (mM) | | | | | | | |
|---|---|---|---|---|---|---|---|---|
| | Mon | I3P | MP | Trp | Ala | Pry | Hmo | HMG |
| Feed S9 | 19.23 | 32.10 | 43.83 | 41.20 | 16.20 | 13.33 | 45.77 | 4.77 |
| Permeate S33 | 0.20 | 1.70 | 10.13 | 6.10 | 0.30 | 0.20 | 5.37 | 0.10 |
| Retentate S32 | 85.90 | 116.20 | 10.40 | 44.93 | 55.17 | 50.73 | 60.20 | 13.00 |
| Recovery Rinse | 4.70 | 6.70 | 0.50 | 3.03 | 2.80 | 2.60 | 3.80 | 0.83 |
| S34 | | | | | | | | |
| Total Recovered | | | | | | | | |
| Fraction | | | | | | | | |
| [S34 + S32] | | | | | | | | |
| Extra Rinse 1 S35 | 0.60 | 0.80 | 0.00 | 0.50 | 0.40 | 0.47 | 0.60 | 0.10 |
| Extra Rinse 2 S36 | 0.10 | 0.00 | 0.00 | 0.10 | 0.10 | 0.10 | 0.00 | 0.00 |
| | Total Quanity (mmol) | | | | | | | |
| | Mon | I3P | MP | Trp | Ala | Pry | Hmo | HMG |
| Feed S9 | 508.04 | 847.90 | 1157.83 | 1088.27 | 427.91 | 352.19 | 1208.90 | 125.91 |
| Permeate S33 | 19.59 | 166.54 | 992.73 | 597.60 | 29.39 | 19.59 | 525.75 | 9.80 |
| Retentate S32 | 453.92 | 614.04 | 54.96 | 237.44 | 291.52 | 268.09 | 318.12 | 68.70 |
| Recovery Rinse | 47.08 | 67.11 | 5.01 | 30.38 | 28.05 | 26.04 | 38.06 | 8.35 |
| S34 | | | | | | | | |
| Total Recovered | | | | | | | | |
| Fraction | | | | | | | | |
| [S34 + S32] | | | | | | | | |
| Extra Rinse 1 S35 | 3.62 | 4.83 | 0.00 | 3.02 | 2.41 | 2.82 | 3.62 | 0.60 |
| Extra Rinse 2 S36 | 1.21 | 0.00 | 0.00 | 1.21 | 1.21 | 1.21 | 0.00 | 0.00 |
| | Total Relative Quanity Recovered (%) | | | | | | | |
| | Mon | I3P | MP | Trp | Ala | Pry | Hmo | HMG |
| Feed S9 | 100.00% | 100.00% | 100.00% | 100.00% | 100.00% | 100.00% | 100.00% | 100.00% |
| Permeate S33 | 3.86% | 19.64% | 85.74% | 54.91% | 6.87% | 5.56% | 43.49% | 7.78% |
| Retentate S32 | 89.35% | 72.42% | 4.75% | 21.82% | 68.13% | 76.12% | 26.31% | 54.56% |
| Recovery Rinse | 9.27% | 7.92% | 0.43% | 2.79% | 6.55% | 7.39% | 3.15% | 6.63% |
| S34 | | | | | | | | |
| Total Recovered | 98.62% | 80.33% | 5.18% | 24.61% | 74.68% | 83.52% | 29.46% | 61.19% |
| Fraction | | | | | | | | |
| [S34 + S32] | | | | | | | | |
| Extra Rinse 1 S35 | 0.71% | 0.57% | 0.00% | 0.28% | 0.56% | 0.80% | 0.30% | 0.48% |
| Extra Rinse 2 S36 | 0.24% | 0.00% | 0.00% | 0.11% | 0.28% | 0.34% | 0.00% | 0.00% |

24 kg feed liquor ((S0) i.e. the permeate from the ultrafiltration step) was treated on a Sepro NF-5 membrane to a concentration factor of 2.5×. After that, diafiltration water was added to the feed tank in order to maintain a constant volume. A diafiltration equivalent would be the volume of the retentate at a 2.5× concentration factor. In this case, it is approximately equal to 9.6 L. Samples were taken after each diafiltration equivalent volume was permeated from the retentate. A total of eight (8) diafiltrations were performed. After the 8^{th} diafiltration equivalent volume, the retentate was concentrated further to a 4.4× factor basis the original UF permeate volume. At completion, the permeate (S33) and the retentate (S32) were drained, weighted and analyzed. Subsequently, 9.9 kg water was recirculated in the membrane during 15 minutes to recover the components from the void volume of the membrane and possibly adsorbed by the membrane. After 15 minutes, the liquor with the “extracted” remaining components (S34) was drained, weighted and analyzed. The overall concentration factor of this combined fraction is 4.3×.

Subsequently, another 6.08 kg of water was pumped through the membrane to check if more components from the void volume of the membrane and adsorbed by the membrane can be recovered. After 15 minutes, the liquor with the “extra extracted” remaining components (S35) was drained, weighted and analyzed. Thereafter, another 11.94 kg of water was recirculated in the membrane during 15 minutes to check if more components from the void volume of the membrane and possibly adsorbed by the membrane can be recovered. After 15 minutes, the liquor with the “extra extracted” remaining components (S36) was drained, weighted and analyzed. Table 8 shows the detailed results for the 8 components in the feed, permeate and retentate from Trial FW-242-E14-N.

TABLE 8a
Concentrations for the Diafiltration Runs for Example FW-242E14-N, NF-5
Membrane at a Feed Pressure of 300 PSI (through 8 diafiltrations)

| | Monatin | Trp | Pyruvate | I3P | MP | HMO | Ala. | HMG |
|---|---|---|---|---|---|---|---|---|
| feed | 508.04 | 847.90 | 1157.83 | 1088.27 | 427.91 | 352.19 | 1208.90 | 125.91 |
| retentate 2.5× | 432.23 | 725.84 | 854.51 | 910.02 | 405.60 | 323.45 | 777.18 | 71.88 |
| retentate at 1D | 476.16 | 738.33 | 578.33 | 834.72 | 438.56 | 355.67 | 786.53 | 88.68 |
| retentate at 2D | 461.29 | 743.98 | 340.95 | 644.66 | 387.75 | 316.12 | 614.10 | 76.40 |
| retentate at 3D | 458.03 | 732.46 | 257.04 | 603.94 | 407.78 | 338.21 | 538.23 | 75.37 |
| retentate at 4D | 474.96 | 749.68 | 177.02 | 503.98 | 381.13 | 320.19 | 501.08 | 77.39 |
| retentate at 5D | 477.50 | 737.07 | 142.38 | 476.53 | 396.14 | 335.12 | 460.06 | 77.48 |
| retentate at 6D | 456.28 | 700.34 | 91.64 | 369.46 | 336.66 | 290.36 | 388.76 | 70.42 |
| etentate at 7D | 452.72 | 678.12 | 58.76 | 289.94 | 289.94 | 254.30 | 357.36 | 70.32 |
| retentate at 8D | 490.78 | 737.61 | 53.78 | 300.62 | 329.43 | 291.97 | 344.80 | 72.99 |
| Final retentate | 453.92 | 614.04 | 54.96 | 237.44 | 291.52 | 268.09 | 318.12 | 68.70 |
| recirc | 47.08 | 67.11 | 5.01 | 30.38 | 28.05 | 26.04 | 38.06 | 8.35 |
| if recombined | 501.00 | 681.15 | 59.97 | 267.83 | 319.57 | 294.14 | 356.18 | 77.04 |
| | Monatin | Trp | Pyruvate | I3P | MP | HMO | Ala. | HMG |

Where all data in Table 8a is expressed in units of millimoles.

TABLE 8b
Alpha values for Example 242-E14-N, NF-5
Membrane at a Feed Pressure of 300 PSI

| DIAFILTRATION | ALPHA | ALPHA | ALPHA | ALPHA |
|---|---|---|---|---|
| RUN | I3P | TRYP | PYRUVATE | ALANINE |
| 2.5X | 15.06 | 3.91 | 45.42 | 14.82 |
| 1D | 18.82 | 4.68 | 61.34 | 18.16 |
| 2D | 23.61 | 5.17 | 78.02 | 21.53 |
| 3D | 29.20 | 6.25 | 98.01 | 28.08 |
| 4D | 26.86 | 5.07 | 87.20 | 23.70 |
| 5D | 25.05 | 4.86 | 78.81 | 22.83 |
| 6D | 27.79 | 4.56 | 92.11 | 19.95 |
| 7D | 59.34 | 8.01 | 200.33 | 36.74 |
| 8D | 55.51 | 8.65 | 173.38 | 39.86 |
| 4.3X | 58.31 | 6.28 | 418.49 | 38.29 |

### EXEMPLARY EMBODIMENTS

A. A method of recovering monatin from a mixture including monatin and at least one of I3P, tryptophan, pyruvate and monatin precursor, comprising pumping the mixture across a membrane having a zeta potential of from −19.0 to −6.0 and wherein greater than 95% of the monatin is rejected by the membrane.
B. The method of embodiment A, wherein the mixture includes monatin and I3P.
C. The method of embodiment B, where the membrane produces an α value greater than 15 for I3P at operating conditions of 15° C.
D. The method of embodiment C, wherein the a value is from about 15.5 to 18.4.
E. The method of embodiment B, wherein the membrane produces an α value greater than 30 for I3P at operating conditions of 30° C.
F. The method of embodiment E, wherein the a value is from about 33.2 to 42.7.
G. The method of embodiment A, wherein the mixture includes monatin and pyruvate.
H. The method of embodiment G, where the membrane produces an α value greater than 30 for pyruvate at operating conditions of 15° C.
I. The method of embodiment H, wherein the a value is from about 34.2 to 35.3.
J. The method of embodiment G, where the membrane produces an α value greater than 30 for pyruvate at operating conditions of 30° C.
K. The method of embodiment J, wherein the a value is from about 54.8 to 62.5.
L. The method of embodiment A, wherein the mixture includes monatin and tryptophan.
M. The method of embodiment L, where the membrane produces an α value greater than 5 for tryptophan at operating conditions of 15° C.
N. The method of embodiment M, wherein the a value is from about 5.6 to 5.8.
O. The method of embodiment L, where the membrane produces an α value greater than 15 for tryptophan at operating conditions of 30° C.
P. The method of embodiment 0, wherein the a value is from about 15.3 to 16.2.
Q. The method of embodiment A, wherein the membrane is a DL type membrane.
R. The method of embodiment A, wherein the membrane is a NF5 type membrane.
S. The method of embodiment A, wherein the mixture includes non-monatin components and wherein diafiltration water is used to further permeate the non-monatin components.
T. A method of recovering monatin from a mixture including monatin, tryptophan and I3P, the method comprising:
passing the mixture through a nanofiltration membrane to retain a second mixture having a lower concentration of I3P and tryptophan than in the first mixture wherein the membrane has a zeta potential of from about −19 to −6 and wherein greater than 95% of the monatin is rejected by the membrane.
U. The method of embodiment T, further comprising adding the second mixture to a dynamic axial compression (DAC) chromatography column packed with a reverse phase resin, wherein the second mixture has a ratio of monatin to I3P greater than about 2;
passing an eluent through the DAC chromatography column to elute monatin from the second mixture; and
collecting at least one fraction from the DAC chromatography column containing monatin having at least about 90% purity.
V. The method of embodiment U, wherein at least about 80% by weight of the monatin in the second mixture is recovered in the at least one fraction from the DAC chromatography column.
W. The method of embodiment V, wherein the second mixture has a ratio of monatin to I3P greater than about 3.22.
X. The method of embodiment V, wherein the second mixture has a ratio of monatin to I3P greater than about 5.

What is claimed is:

1. A method of recovering monatin from a mixture including monatin and at least one of I3P, tryptophan, pyruvate and monatin precursor, comprising pumping the mixture across a membrane having a zeta potential of from −19.0 to −6.0 and wherein greater than 90% of the monatin is rejected by the membrane.

2. The method of claim 1, wherein the mixture includes monatin and I3P.

3. The method of claim 2, where the membrane produces an α value greater than 15 for I3P at operating conditions of 15° C.

4. The method of claim 2, wherein the membrane produces an α value greater than 30 for I3P at operating conditions of 30° C.

5. The method of claim 1, wherein the mixture includes monatin and pyruvate.

6. The method of claim 5, where the membrane produces an α value greater than 30 for pyruvate at operating conditions of 15° C.

7. The method of claim 5, where the membrane produces an α value greater than 30 for pyruvate at operating conditions of 30° C.

8. The method of claim 1, wherein the mixture includes monatin and tryptophan.

9. The method of claim 8, where the membrane produces an α value greater than 5 for tryptophan at operating conditions of 15° C.

10. The method of claim 8, where the membrane produces an α value greater than 15 for tryptophan at operating conditions of 30° C.

11. The method of claim 1, wherein the membrane is a DL type membrane.

12. The method of claim 1, wherein the membrane is a NF5 type membrane.

13. The method of claim 1, wherein the mixture includes non-monatin components and wherein diafiltration water is used to further permeate the non-monatin components.

14. A method of recovering monatin from a mixture including monatin, tryptophan and I3P, the method comprising:
passing the mixture through a nanofiltration membrane to retain a second mixture having a lower concentration of I3P and tryptophan than in the first mixture wherein the membrane has a zeta potential of from about −19 to −6 and wherein greater than 90% of the monatin is rejected by the membrane.
